(19) United States
(12) Patent Application Publication
(10) Pub. No.: US 2005/0075709 A1
(43) Pub. Date: Apr. 7, 2005

(54) Biomedical electrode of enhanced surface area

(75) Inventors: Kenneth R. Brennen, Sioux Falls, SD (US); William J. Schindeldecker, Foreston, MN (US); Scott J. Brabec, Elk River, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/368,153

(22) Filed: Feb. 18, 2003

(51) Int. Cl. A61N 1/05

(52) U.S. Cl. 607/122

(57) ABSTRACT

An implantable biomedical electrode includes platinum particles electrochemically deposited upon, and sintered to a surface.

### FIELD OF THE INVENTION

The present invention is related to biomedical, implantable electrical leads and electrodes. More specifically, the present invention is related to implantable electrodes including sintered platinum black particles formed on a metallic substrate.

### BACKGROUND OF THE INVENTION

During the delivery of a pacing stimulus an electrochemical reaction at the electrode-tissue interface site occurs. This effect is commonly termed polarization impedance. Polarization impedance refers to the degree of polarization at the electrode-tissue interface, which can impact efficiency of the electrode. The electrode's efficiency in transferring energy to or from adjacent body tissue is affected by the polarization impedance.

A low polarization impedance is important in stimulation electrodes because the energy required to stimulate the heart is reduced by having a low polarization impedance electrode. A low polarization impedance electrode is also desirable for sensing electrodes as the low polarization electrode reduces the demands placed on the input impedance of the sensing amplifier. This acts to increase the strength of sensed of biomedical electrical signals.

Some electrodes display some degree of polarization following a stimulation pulse. This polarization dissipates following the stimulation pulse and is not believed to significantly interfere with the delivery of the following stimulation pulses. However, polarization of these electrodes may be sufficient to interfere with the ability of the electrode to respond to the electrical activity of the heart during the period immediately following delivery of the stimulation impulse. It is therefore desirable to have a stimulation electrode polarization sufficiently low so as to be able to sense heart activity immediately following the stimulation pulse. This is particularly important for sensing the response of the myocardium evoked by a pacing or defibrillation pulse.

Following implant, the chronic stimulation threshold is two to three times higher than the acute stimulation threshold initially observed. A larger stimulation threshold requires a corresponding increase in the required amount of energy, reducing the efficiency of the implanted device. The threshold increase is normally attributed to a fibrous capsule that develops around the electrode tip. Specifically, the development of a layer or layers of unexcitable connective tissue surrounds the electrode tip at the stimulation site. The fibrous capsule results in a virtual electrode surface area that is considerably greater than the actual surface of the electrode. The increase in surface area lowers current densities at the interface with excitable tissue and results in a higher stimulation threshold. The microstructure of the electrode surface is one factor impacting the thickness of the fibrous capsule.

Polished platinum and platinum-iridium substrates serve as better electrodes when coated with other materials that increase the effective surface area of the substrates. However, the polished platinum or platinum-iridium substrates often do not allow these particles to optimally adhere to the surface.

Many electrodes currently are based upon a platinum-iridium (90:10) substrate, having platinum black particles electrochemically deposited on the platinum-iridium substrate. The platinum black particles can have a very small, for example, less than one micron, mean particle diameter. The small particle diameter significantly increases the effective surface area of the electrode, thus providing decreased polarization impedance.

Current methods include electrochemically depositing platinum black upon a platinum-iridium substrate, leaving the platinum-iridium substrate covered with small, variegated, fragile, fractal surface features. Such methods are described in U.S. Pat. No. 4,502,492, incorporated herein by reference. The more fragile structures can be removed from the substrate surface by applying a brush to the surface. Current manufacturing techniques include manually inspecting the electrode surface under a microscope, and hand brushing the surface to dislodge the more fragile and/or more weakly adhered platinum black particles from the surface.

### BRIEF DESCRIPTION OF THE DRAWINGS

FIG. 1 is a plan view of a lead.

FIG. 2 is a plan view of an alternate tip electrode.

FIG. 3 is a schematic of an enlarged partial section through an electrode, according to one embodiment of the present invention.

FIG. 4 is a schematic of an enlarged partial section through an electrode, according to another embodiment of the present invention.

FIG. 5 is a microphotograph of a polished platinum-iridium surface, magnified 2500× and having a 10-micron bar to indicate scale.

FIG. 6 is a microphotograph of a surface, such as that illustrated in FIG. 5, on which platinum black particles have been electrochemically deposited, magnified 2500× and having a 12-micron bar to indicate scale.

FIG. 7 is a microphotograph of the surface of FIG. 6, magnified 20,000× and having a 1.5-micron bar to indicate scale.

FIG. 8 is a microphotograph of a surface, such as that illustrated in FIGS. 6 and 7, on which platinum black particles have been sintered, magnified 2500× and having a 12-micron bar to indicate scale.

FIG. 9 is a microphotograph of the surface of FIG. 8, magnified 20,000× and having a 1.5-micron bar to indicate scale.

FIG. 10 is a microphotograph of a surface, such as that illustrated in FIGS. 6 and 7, on which platinum black particles have been sintered, magnified 2500× and having a 12-micron bar to indicate scale.

FIG. 11 is a microphotograph of the surface of FIG. 10, magnified 20,000× and having a 1.5-micron bar to indicate scale.

FIG. 12 is a microphotograph of a surface, such as that illustrated in FIGS. 6 and 7, on which platinum black particles have been sintered, magnified 2500× and having a 10-micron bar to indicate scale.

FIG. 13 is a microphotograph of the surface of FIG. 12, magnified 20,000× and having a 2-micron bar to indicate scale.

FIG. 14 is a microphotograph of a surface, such as that illustrated in FIGS. 6 and 7, on which platinum black particles have been sintered, magnified 2500× and having a 12-micron bar to indicate scale.

FIG. 15 is a microphotograph of the surface of FIG. 14, magnified 20,000× and having a 1.5-micron bar to indicate scale.

FIG. 16 is a microphotograph of a surface, such as that illustrated in FIGS. 8 and 9, on which a second layer of platinum black particles has been electrochemically deposited over a sintered layer of platinum black particles, magnified 2500× and having a 12-micron bar to indicate scale.

FIG. 17 is a microphotograph of the surface of FIG. 16, magnified 20,000× and having a 1.5-micron bar to indicate scale.

### DETAILED DESCRIPTION OF THE DRAWINGS

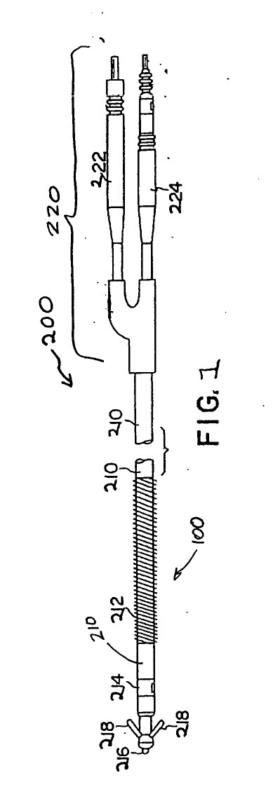

FIG. 1 is a plan view of a defibrillation lead 200 including an elongated insulative lead body 210, preferably fabricated of silicone rubber, polyurethane or other biocompatible polymer. As illustrated in FIG. 1, a proximal end of lead body 210 is terminated in a bifurcated connector assembly 220, such as is commonly known to those skilled in the art, including an IS-1 connector leg 224 and a DF-1 connector leg 222, by which connector legs lead 200 is electrically connected to an implantable medical device (not shown). As further illustrated in FIG. 1, a distal end 100 of lead body 210 carries a defibrillation electrode coil 212, a ring electrode 214, and a tip electrode 216, each electrode coupled to a connector leg, via an insulated conductor within lead body 210, in a manner commonly known to those skilled in the art. Tines 218 are provided for maintaining tip electrode 216 in contact with myocardial tissue. According to various embodiments of the present invention any one or all of the aforementioned electrodes include an enhanced surface formed, in part, by sintering electrochemically deposited platinum black particles. Further, although lead 200 includes various types of electrodes, alternate embodiments according to the present invention need only include one type of electrode having an enhanced surface as described herein.

FIG. 2 is a plan view of an alternate tip electrode. FIG. 2 illustrates an alternate tip electrode as a helix electrode 104, terminating distal end 100 of lead body 210, thus replacing tines 218 and tip electrode 216 illustrated in FIG. 1. According to an embodiment of the present invention helix electrode 104 includes an enhanced surface formed, in part, by sintering electrochemically deposited platinum black particles.

According to embodiments of the present invention, coil electrode 212, ring electrode 214, tip electrode 216, helix electrode 104, or any other form of electrode suitable for mounting on a lead, such as lead 200, are formed from a material including platinum, for example an alloy of platinum and iridium, and have a surface enhanced by sintering electrochemically deposited platinum black particles.

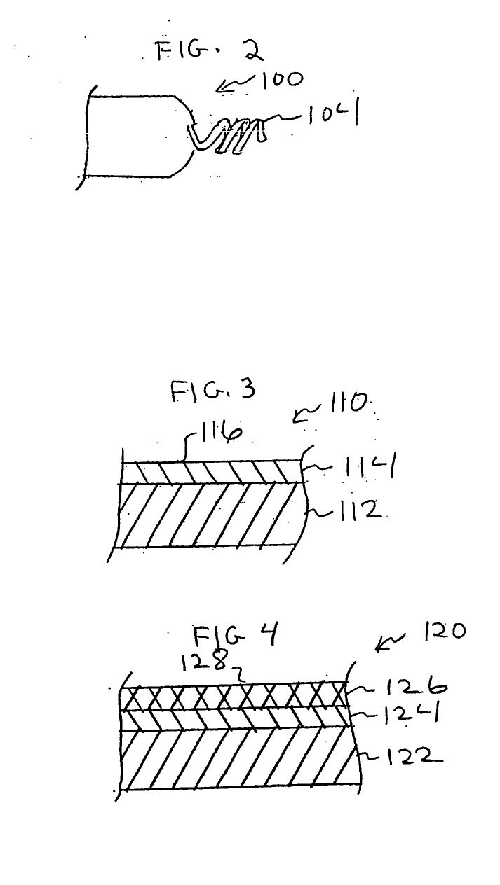

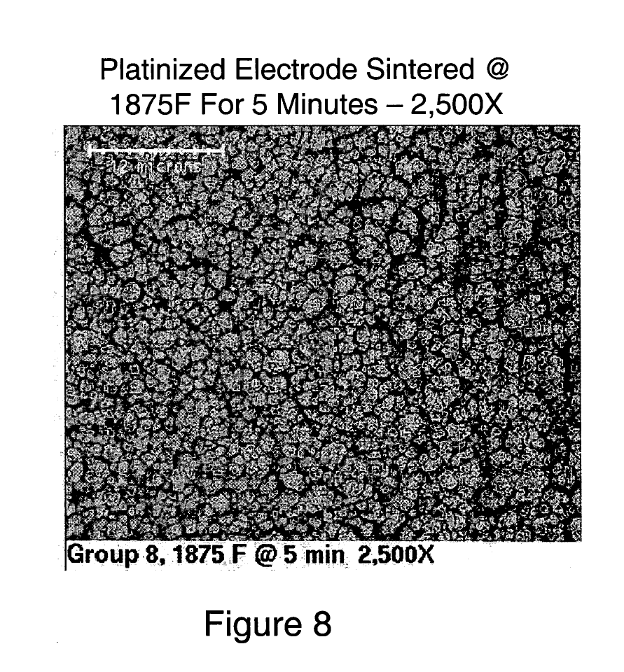

FIG. 3 is a schematic of an enlarged partial section through an electrode 110 including an enhanced surface 116. As illustrated in FIG. 3, electrode 110 includes a substrate 112 having a first layer 114 formed thereover. According to the present invention, substrate 112 is comprised of platinum, for example a platinum-iridium alloy, and first layer 114 is an electrochemically deposited platinum black layer that has been sintered. Surface 116 is exposed having an enhanced surface area in one embodiment of an electrode according to the present invention. Alternately, surface 116 serves foundation for another layer as illustrated in FIG. 4. Examples of surface 116 are presented in FIGS. 8-15.

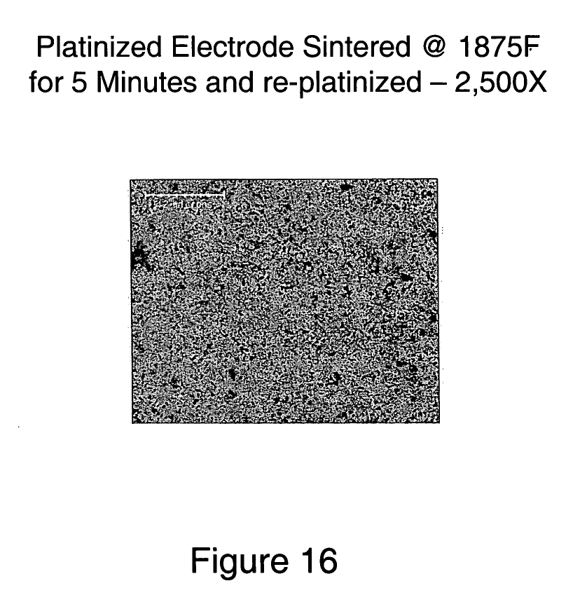

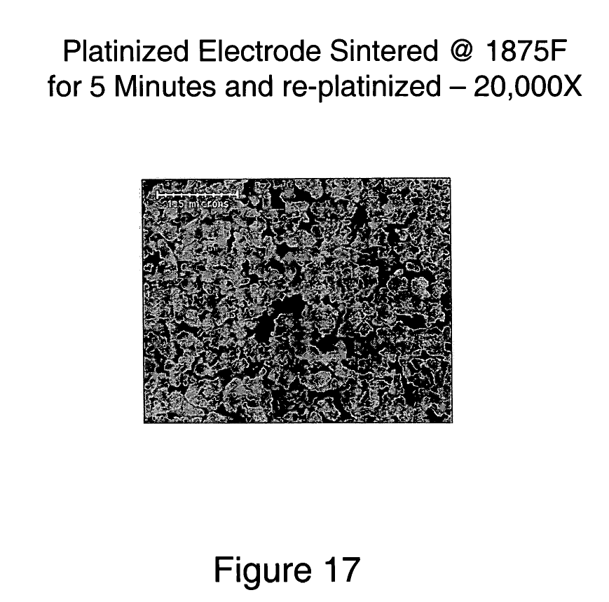

FIG. 4 is a schematic of an enlarged partial section through an electrode 120 including an enhanced surface 128. As illustrated in FIG. 4, electrode 120 includes a substrate 122 having a first layer 124 and a second layer 126 formed thereover. According to the present invention, substrate 122 is comprised of platinum, for example a platinum-iridium alloy, and first layer 124 is an electrochemically deposited platinum black layer that has been sintered. Second layer 126 is a material forming enhanced surface 128 whose adhesion to substrate 122 is enhanced by first layer 124. In one group of embodiments, second layer 126 is selected from the group consisting of iridium-oxide, titanium-nitride, ruthenium-oxide, platinum black particles, and combinations thereof. An example of surface 128 is presented in FIGS. 16 and 17.

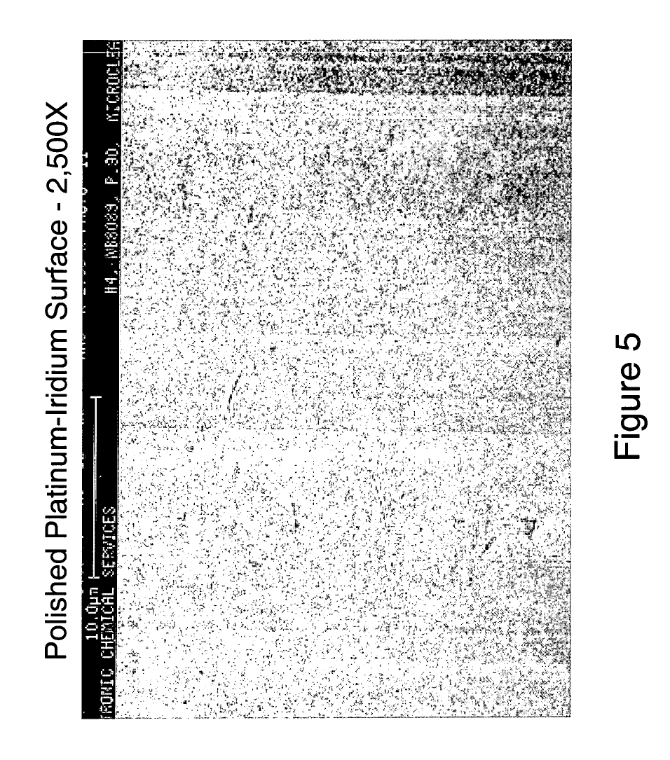

FIG. 5 is a microphotograph of a polished platinum-iridium surface, magnified 2500× and having a 10-micron bar to indicate scale. The substrate is part of a 90:10 platinum-iridium anode ring, having a geometric surface area between approximately 36 square millimeters and approximately 38 square millimeters, that has been acid etched in aqua-regia for 10 minutes, followed by rinses in de-ionized water and isopropyl alcohol. The polished platinum-iridium surface, by itself, serves as a less-than-optimal electrode, and preferably is further enhanced.

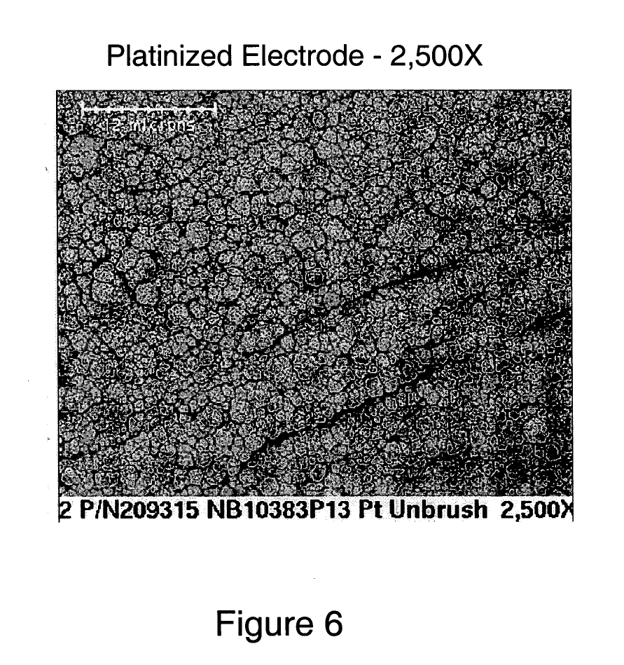

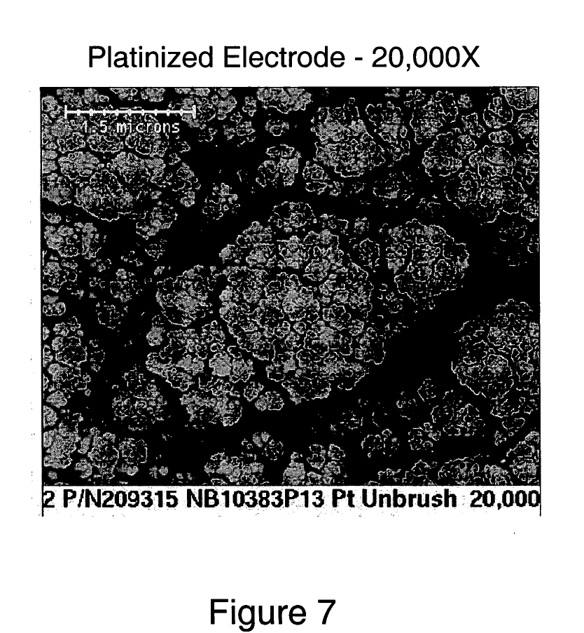

FIG. 6 is a microphotograph of an electrode surface, such as that illustrated in FIG. 5, on which platinum black particles have been electrochemically deposited, magnified 2500× and having a 12-micron bar to indicate scale. FIG. 7 is a microphotograph of the surface of FIG. 6, magnified 20,000× and having a 1.5-micron bar to indicate scale. According to the present invention, a platinization process, alternately described herein as electrochemical deposition, is used to create the surface of FIGS. 6 and 7. The platinization process includes an electro-chemical deposition of platinum black particles in a chloroplatinic acid bath. One supplier of chloroplatinic acid is Johnson-Matthey. In the example illustrated in FIGS. 6 and 7, electro-chemical deposition was continued for 2 minutes at a current of 4.5 milliamps. The electrodes were then rinsed in de-ionized water, followed by isopropyl alcohol rinses. Aforementioned U.S. Pat. No. 4,502,492 describes a platinization process in greater detail.

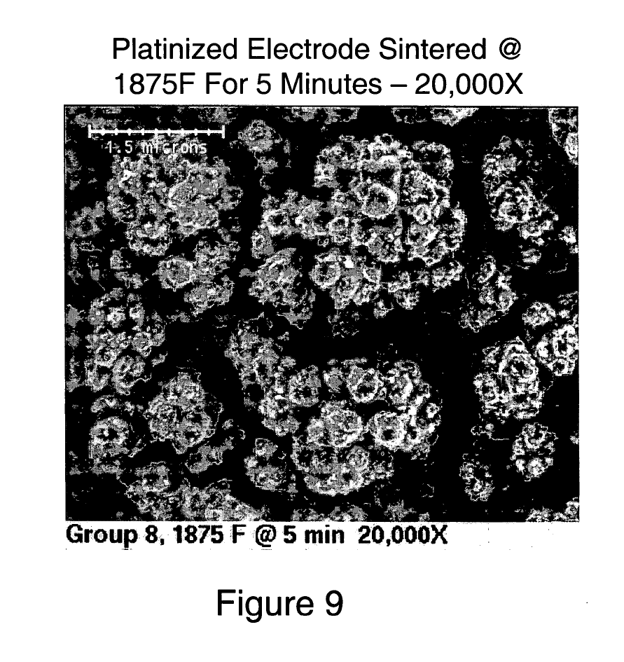

FIG. 8 is a microphotograph of an electrode surface, such as that illustrated in FIGS. 6 and 7, on which platinum black particles have been sintered, magnified 2500× and having a 12-micron bar to indicate scale. FIG. 9 is a microphotograph of the surface of FIG. 8, magnified 20,000× and having a 1.5-micron bar to indicate scale. As used in the present application, the term “sintering” or “sintered” refers to a process in which the particles are heated to a temperature sufficient to at least partially re-flow the particles. According to the present invention, sintering forms enhanced adhesion of platinum black particles to an underlying substrate, and in some embodiments of the present invention, platinum black particles may also adhere to, and coalesce with adjacent particles during sintering. The sintering process as illustrated by the example of FIGS. 8 and 9, and the remaining figures, is preferably carried out in a sintering furnace under a vacuum of at least 10×10⁻⁵ Torr. FIGS. 8 and 9 illustrate modified platinized surfaces, according to one embodiment of the present invention, formed by sintering a temperature of 1875° F. for five minutes. FIG. 9 may be compared to FIG. 7 to observe the change in surface features caused by sintering. The sintered surface shown in FIGS. 8 and 9 is highly irregular having an effective active surface area between that of a smooth surface, such as that illustrated in FIG. 5 and a fractally-coated surface, such as that illustrated in FIGS. 6 and 7.

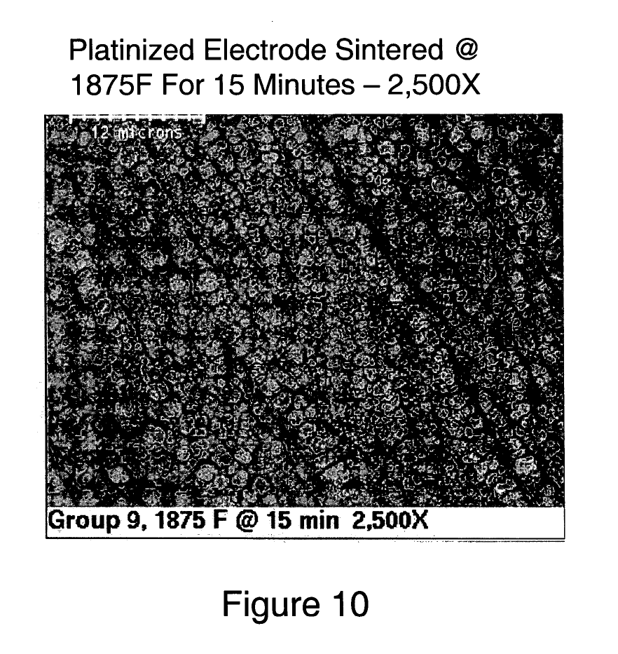

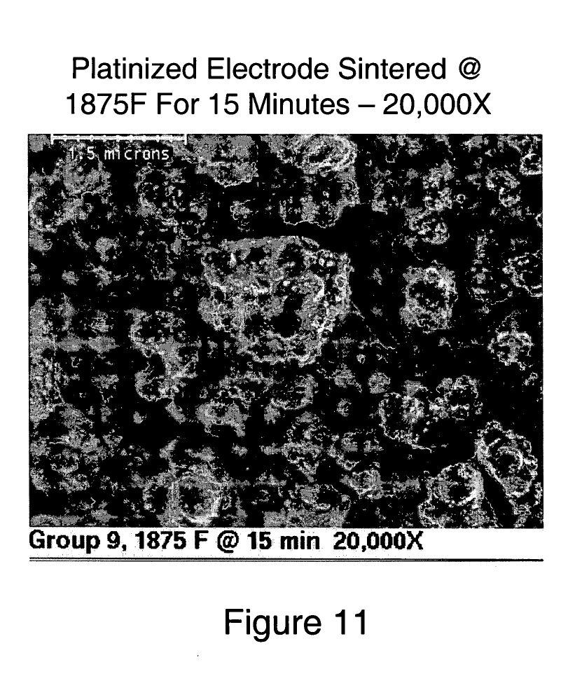

FIG. 10 is a microphotograph of an electrode surface, such as that illustrated in FIGS. 6 and 7, on which platinum black particles have been sintered, magnified 2500× and having a 12-micron bar to indicate scale. FIG. 11 is a microphotograph of the surface of FIG. 10, magnified 20,000× and having a 1.5-micron bar to indicate scale. FIGS. 10 and 11 illustrate modified platinized surfaces, according to one embodiment of the present invention, formed by sintering a temperature of 1875° F. for fifteen minutes. FIG. 11 may be compared with FIG. 7 to observe the change in surface features caused by sintering, and compared with FIG. 9 to observe the change in features caused by sintering for a longer period of time.

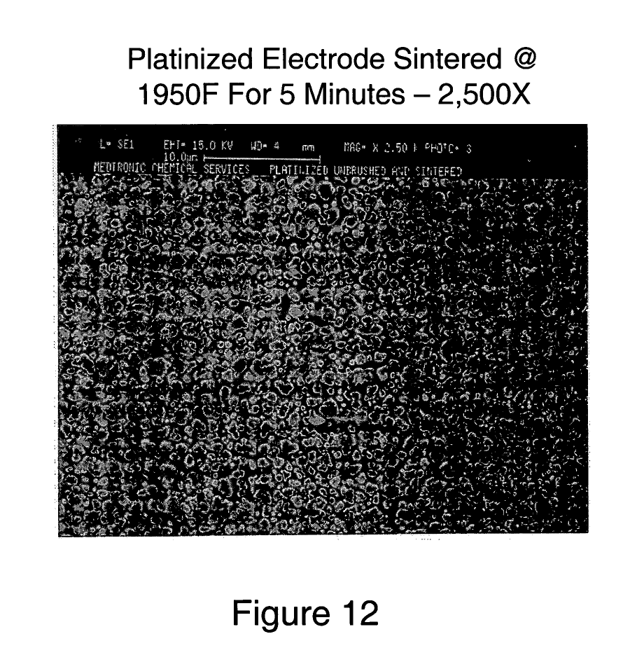

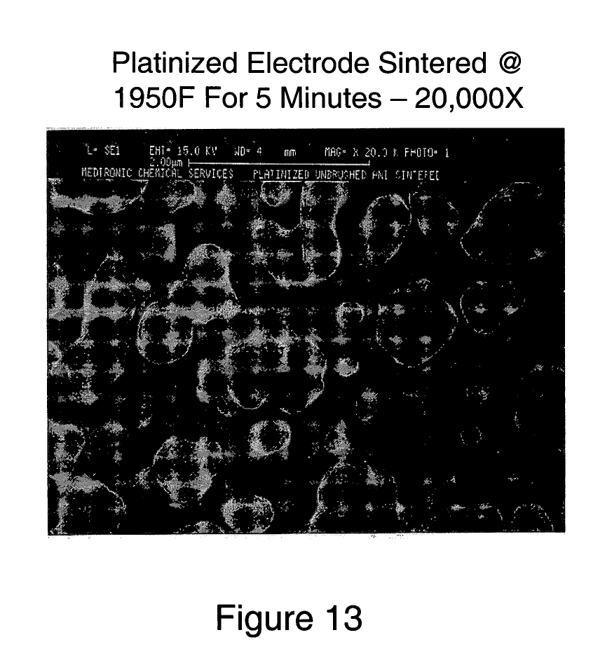

FIG. 12 is a microphotograph of an electrode surface, such as that illustrated in FIGS. 6 and 7, on which platinum black particles have been sintered, magnified 2500× and having a 10-micron bar to indicate scale. FIG. 13 is a microphotograph of the surface of FIG. 12, magnified 20,000× and having a 2-micron bar to indicate scale. FIGS. 12 and 13 illustrate modified platinized surfaces, according to one embodiment of the present invention, formed by sintering at a temperature of 1950° F. for five minutes. FIG. 13 may be compared with FIG. 7 to observe the change in surface features caused by sintering, and compared with FIG. 9 to observe the change in features caused by sintering at a higher temperature.

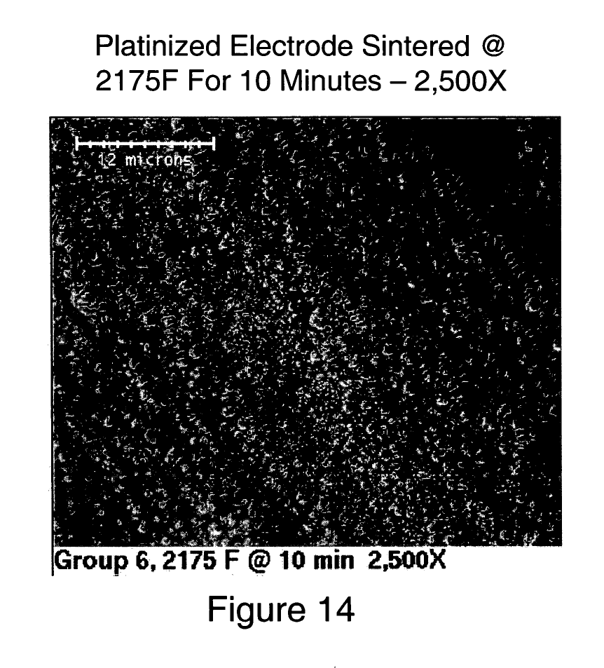

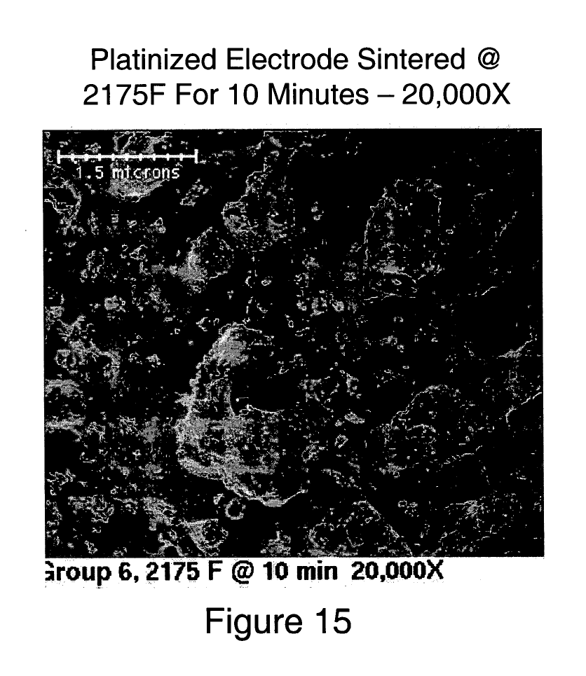

FIG. 14 is a microphotograph of an electrode surface, such as that illustrated in FIGS. 6 and 7, on which platinum black particles have been sintered, magnified 2500× and having a 12-micron bar to indicate scale. FIG. 15 is a microphotograph of the surface of FIG. 14, magnified 20,000× and having a 1.5-micron bar to indicate scale. FIGS. 14 and 15 illustrate modified platinized surfaces, according to one embodiment of the present invention, formed by sintering at 2175° F. for 10 minutes. FIG. 15 may be compared to FIG. 7 to observe the change in surface features caused by sintering, and compared to FIGS. 9, 11, and 13 to observe the change in features caused by sintering at a higher temperature.

According to the present invention, as is illustrated by the above examples, sintering changes a delicate platinized surface to a more robust surface. The heating, causing at least a partial re-flow of platinum black particles, strengthens the adhesion of the particles to both the substrate and creates a more durable foundation for a subsequent layer. Examples of subsequent layers include iridium-oxide, ruthenium-oxide, titanium-nitride, platinum black particles, and combinations thereof. According to some embodiments of the present invention, iridium-oxide or ruthenium-oxide are either deposited via sputtering or via thermal decomposition in a slurry, both methods being known and understood by those skilled in the art. According to another embodiment of the present invention titanium-nitride is deposited via sputtering and, according to yet another embodiment of the present invention, another layer of platinum black particles are electrochemically deposited on the sintered surface.

FIG. 16 is a microphotograph of an electrode surface, such as that illustrated in FIGS. 8 and 9, on which a second layer of platinum black particles has been electrochemically deposited over a sintered layer of platinum black particles, magnified 2500× and having a 12-micron bar to indicate scale. FIG. 17 is a microphotograph of the surface of FIG. 16, magnified 20,000× and having a 1.5-micron bar to indicate scale. FIGS. 16 and 17 illustrate one embodiment of the present invention wherein a sintered layer, such as that depicted as first layer 124 in FIG. 4, serves as a foundation for a surface (ie 128 in FIG. 4) created by a second layer (ie 126 in FIG. 4) having an increased microscopic surface area. In the example of FIGS. 16 and 17, the surface is created by platinization, however, in alternate embodiments a second layer of titanium-nitride, iridium-oxide, or ruthenium-oxide creates a different surface structure having enhanced properties to reduce polarization or pacing impedance.

Electrodes according to the present invention can be included in implantable biomedical leads in general. Such leads include, for example, cardiac sensing leads, cardiac pacing leads, cardiac defibrillation leads, neurological stimulation leads, and neurological sensing leads. The present invention may also be usefully employed in interventional catheters such as electrophysiology mapping catheters. It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. A method for making an implantable biomedical electrode, comprising:
electrochemically depositing platinum particles on a metallic substrate comprising platinum; and
heating the substrate and the platinum particles to sinter the platinum particles to the substrate.

2. The method of claim 1, wherein the metallic substrate further comprises iridium.

3. The method of claim 1, wherein a temperature for heating is between approximately 1800 degrees Fahrenheit and approximately 1900 degrees Fahrenheit and is held for between approximately 4 minutes and approximately 10 minutes.

4. The method of claim 1, wherein a temperature for heating is between approximately 1800 degrees Fahrenheit and approximately 1900 Fahrenheit and is held for between approximately 10 minutes and approximately 20 minutes.

5. The method of claim 1, wherein a temperature for heating is between approximately 1900 degrees Fahrenheit and approximately 2200 degrees Fahrenheit and is held for between approximately 4 and approximately 15 minutes.

6. The method of claim 1, further comprising forming a layer over the sintered platinum particles, the layer comprising materials selected from the group consisting of iridium and ruthenium.

7. The method of claim 6, wherein forming the layer includes sputtering.

8. The method of claim 6, wherein forming the layer includes thermal decomposition in a slurry.

9. The method of claim 1, further comprising forming a layer over the sintered platinum particles, the layer comprising titanium-nitride.

10. The method of claim 9, wherein forming the layer includes sputtering.

11. The method of claim 1, further comprising forming a layer over the sintered platinum particles, the layer comprising platinum.

12. The method of claim 11, wherein forming the layer includes electrochemical deposition.

13. An implantable biomedical electrode, comprising a surface and platinum particles electrochemically deposited upon, and sintered to the surface.

14. The electrode of claim 13, wherein the surface is formed by a platinum-iridium alloy.

15. The electrode of claim 13, wherein the platinum particles are sintered to the surface at a temperature of between approximately 1800 degrees Fahrenheit and approximately 1900 degrees Fahrenheit held for a time between approximately 4 minutes and approximately 10 minutes.

16. The electrode of claim 13, wherein the platinum particles are sintered to the surface at a temperature of between approximately 1800 degrees Fahrenheit and approximately 1900 Fahrenheit held for a time between approximately 10 minutes and approximately 20 minutes.

17. The electrode of claim 13, wherein the platinum particles are sintered to the surface at a temperature of between approximately 1900 degrees Fahrenheit and approximately 2200 degrees Fahrenheit held for a time between approximately 4 minutes and approximately 15 minutes.

18. The electrode of claim 13 further comprising a layer formed over the sintered platinum particles, the layer selected from the group consisting of iridium-oxide, ruthenium-oxide, titanium-nitride, and platinum.

19. An implantable biomedical lead comprising an electrode including a surface and platinum particles electrochemically deposited upon, and sintered to the surface.

20. The lead of claim 19, wherein the surface of the electrode is formed by a platinum-iridium alloy.

21. The lead of claim 19, wherein the platinum particles are sintered to the surface of the electrode at a temperature of between approximately 1800 degrees Fahrenheit and approximately 1900 degrees Fahrenheit held for a time between approximately 4 minutes and approximately 10 minutes.

22. The lead of claim 19, wherein the platinum particles are sintered to the surface of the electrode at a temperature of between approximately 1800 degrees Fahrenheit and approximately 1900 Fahrenheit held for a time between approximately 10 minutes and approximately 20 minutes.

23. The lead of claim 19, wherein the platinum particles are sintered to the surface of the electrode at a temperature of between approximately 1900 degrees Fahrenheit and approximately 2200 degrees Fahrenheit held for a time between approximately 4 minutes and approximately 15 minutes.

24. The lead of claim 19, further comprising a layer formed over the sintered platinum particles of the electrode, the layer selected from the group consisting of iridium-oxide, ruthenium-oxide, titanium-nitride, and platinum.

25. The lead of claim 19, wherein the electrode is a coil electrode.

26. The lead of claim 19, wherein the electrode is a ring electrode.

27. The lead of claim 19, wherein the electrode is a tip electrode.

28. The lead of claim 27, wherein the tip electrode is a helix electrode.
